(19) United States
(12) Patent Application Publication
(10) Pub. No.: US 2010/0130972 A1
(43) Pub. Date: May 27, 2010

(54) ELECTRICAL SKIN TREATMENT DEVICE AND METHOD

(75) Inventors: Michael Yambor, Medina, OH (US); Lawrence M. Sears, Hunting Valley, OH (US); Stephanie A.S. Harrington, Mentor, OH (US); Mark A. Williams, Mentor, OH (US); Jennifer S. Innamorato, Twinsburg, OH (US); Thomas J. Pavsek, Kirtland, OH (US)

Correspondence Address: RENNER OTTO BOISSELLE & SKLAR, LLP, 1621 EUCLID AVENUE, NINETEENTH FLOOR, CLEVELAND, OH, 44115, US

(73) Assignee: FRANTZ MEDICAL DEVELOPMENT, LTD., Mentor, OH (US)

(21) Appl. No.: 12/618,818

(22) Filed: Nov. 16, 2009

Related U.S. Application Data
(60) Provisional application No. 61/116,854, filed on Nov. 21, 2008.

(51) Int. Cl. A61B 18/04; A61N 1/00

(52) U.S. Cl. 606/34; 607/2

(57) ABSTRACT

An electrical device for treating problem skin areas, including warts, has an electrode and a power source coupled to the electrode for generating an arc over a gap between a distal end of the electrode and a patient's skin when the electrode is placed in spaced proximity to the patient's skin. The power source provides electricity to the electrode with a frequency of at least 100 kHz, an open-circuit voltage of less than 2 kV_{RMS}, and a total power of less than 2 W.

### FIELD OF THE INVENTION

Our invention relates to an electrical device and method for treating problem skin areas, such as warts and other skin infections, and more particularly to a treatment device and method that applies electric sparks to the problem skin area.

### BACKGROUND

Doctors have been searching for new treatments for problem skin areas, such as warts, for many years. A wart is a viral infection of the skin that creates a thickened area on the skin. Treatments for warts have included cutting and removing a section of flesh around the wart; burning the wart with lasers, heated elements, or chemicals; eroding the wart with acid; and freezing the wart, such as with liquid nitrogen.

Doctors also have used high powered electricity to cut and burn a patient's skin, including for the treatment of warts. Unfortunately, current treatment methods tend to be messy and/or painful, and can do permanent damage to the skin.

### SUMMARY

Our invention provides an electrical device and method that uses a relatively low current and low power to treat problem skin areas, such as an infection in the skin, including but not limited to the treatment of viruses in thickened skin, such as warts.

More particularly, our invention provides an electrical device that includes (a) an electrode, and (b) a power source coupled to the electrode for generating an arc over a gap between a distal end of the electrode and a patient's skin when the electrode is placed in spaced proximity to a patient's skin. The power source can provide electricity to the electrode with a frequency of at least 100 kHz, an open-circuit voltage of less than 2 kV_{RMS}, and a total power of less than 2 W, without a return electrode.

The device also can include one or more of the following features:

(a) a non-electrically-conductive spacer extending beyond the distal end of the electrode for contact with the surface of a patient's skin to define a predetermined gap between a contact surface of the spacer and the distal end of the electrode;

(b) an abrasive surface on the electrode to aid in removal of treated skin;

(c) oscillating means coupled to the electrode to oscillate the distal end of the electrode within a controlled distance along an axis between an extended position and a retracted position removed from the extended position to facilitate maintaining and re-establishing an arc between the surface of the skin and the distal end of the electrode;

(d) a vacuum generator to evacuate air and draw fumes away from the distal end of the electrode and the patient's skin;

(e) an electrode having a length dimension, and the distal end of the electrode is movable relative to a central longitudinal axis;

(f) an electrode that is movable relative to the longitudinal axis to allow the distal end of the electrode to sweep through a larger area;

(g) an electrode where a portion at a distal end of the electrode is offset from a longitudinal axis of another portion of the electrode;

(h) a supply of gas or liquid and an outlet port to direct the fluid toward a distal end of the electrode;

(i) the device in combination with a protective non-electrically-conductive material for placement adjacent a treatment area to protect healthy skin from the electrical arc; and

(j) an electrode that includes an array of electrodes connected to the voltage generator, and a voltage distributor for applying a voltage to more than one electrode in the array.

Our invention also provides a method for treating problem skin areas that includes the following steps: (a) generating a voltage and providing that voltage to a distal end of an electrode, and (b) positioning the electrode in proximity to a patient's skin to form an arcable gap between the electrode and the skin to produce an electric spark that arcs across the gap with sufficient intensity to treat the problem skin area but insufficient to cause significant damage to normal surrounding tissue.

The method can further include one or more of the following steps:

(a) generating a high frequency (at least 100 kHz) voltage (less than 2 kV_{RMS} open circuit) with less than 2 W of power to a monopolar electrode;

(b) providing a voltage such that in the moving step the electric spark arcs across the gap with a substantially constant current of less than 30 mA_{RMS} between the electrode and the skin;

(c) contacting the surface of a patient's skin near a problem skin area with a non-electrically-conductive element that spaces the patient's skin from a distal end of an electrode to form a gap between a contact surface of the non-electrically conductive element and the distal end of the electrode;

(d) generating a vacuum near the patient's skin to evacuate air and draw fumes away from the patient's skin; and

(e) measuring the size of a treatment area of a patient's skin, and selecting a plurality of electrodes to distribute voltage to based on the measured size of the treatment area.

The foregoing and other features of the invention are more fully described and particularly pointed out in the claims. The following description and annexed drawings set forth in detail several illustrative embodiments, these embodiments being indicative of but a few of the various ways in which the principles of the invention may be employed.

### BRIEF DESCRIPTION OF THE DRAWINGS

FIG. 1 is a schematic view of an electrical device provided by our invention.

FIG. 2 is a perspective view of an exemplary electrical device provided by our invention.

FIG. 3 is a schematic elevation view of the electrical device of FIG. 2

FIG. 4 is a partial view of a distal end of an electrical device provided by our invention.

FIG. 5 is a partial view of a distal end of another electrical device provided by our invention.

FIG. 6 is a partial of a distal end of another electrical device provided by our invention.

FIG. 7 is a perspective view of another electrical device provided by our invention.

FIG. 8 is a perspective view of an electrical device similar to the device shown in FIG. 7.

FIG. 9 is a perspective view of an electrical device similar to the device shown in FIG. 7.

FIG. 10 is a partial of a distal end of another electrical device provided by our invention.

FIGS. 11 and 12 are partial end views of another electrical device provided by our invention that illustrate movement of one element.

FIG. 13 is a partial view of an electrical device in combination with a protective sheet provided by our invention.

FIG. 14 is a schematic cross-sectional view of an exemplary removable electrode assembly unit mountable to a distal end of another electrical device provided by our invention.

### DETAILED DESCRIPTION

Our invention provides an electrical device and method that uses a relatively low current and low power to treat problem skin areas. Problem skin areas can be caused by, among other things, an infection in the skin, including but not limited to viruses, such as the viruses that cause warts. We have found that in proper use a device employing our invention can effectively and nearly painlessly treat some problem skin areas, and is particularly effective in treating warts, a treatment that has been sought for many years without success.

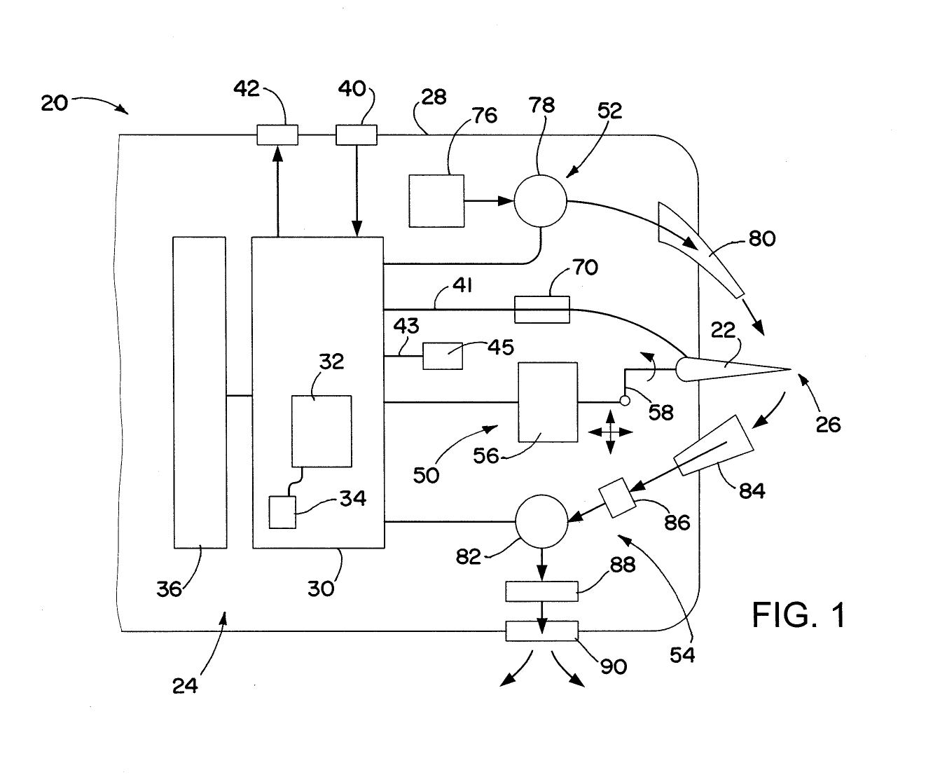

Turning now to the drawings, and initially FIG. 1, our invention provides an electrical device 20 that includes at its core an electrode 22 and a power source 24 coupled to the electrode 22 for generating an arc over a gap G (FIG. 3) between a distal end 26 of the electrode 22 and a patient's skin S (FIG. 3) when the electrode 22 is placed in spaced proximity to a patient's skin. The power source 24 preferably, although not necessarily, is contained in a housing 28 from which the electrode 22 extends. The electrode 22 has a length dimension and a longitudinal axis generally along its length. The electrode 22 can be a disposable component, or it can be removable for cleaning and sterilization, or replaceable with a different type of electrode. Several different types of electrodes are described below, but other variations in shape and materials are envisioned. The electrode 22 provides an electrically-conductive path for the electricity provided by the power source 24 and typically has a pointed distal end 26 at which an electrical charge can accumulate to create a spark that will arc across the gap.

The power source 24 generally includes a controller 30 with a processor 32 and a memory 34 coupled to the processor 32 for storing any programming required for generating, monitoring, and/or regulating the necessary electrical voltage provided to the electrode 22, and a supply of electricity 36 controlled by the controller 30. The supply of electricity can include any source of electricity, including an electricity generator, or a power cord for connection to an electrical outlet, and a suitable transformer (not shown). The power source 24 is preferably self-contained within the device 20, if not within a common insulated housing 28, so that it can be portable and used unencumbered by a power cord. For example, the supply of electricity 36 can include a single-use or rechargeable battery, a fuel cell, or the like. The power source 24 also can include an input device 40, such as an on/off switch, and an output device 42, such as a status-indicator light, for providing information to and from the controller 30.

An exemplary power source 24, which also could be referred to or include a voltage generator or voltage-generating means, has its output terminal 41 connected to the electrode 22 and provides it with an open circuit voltage of less than 2 kilovolts RMS (2 kV_{RMS}), at a frequency of at least one hundred kilohertz (100 kHz), and a total power of less than two watts (2 W). The other output terminal 43 of the power source 24 is connected to a small metallic plate 45 which is contained within the common insulated housing 28. This metallic plate 45 provides capacitive coupling between the power source 24 and the body of the operator of the device. In turn, the operator is capacitively coupled to ground. Since the patient is also capacitively coupled to ground, a complete circuit is available without the need for a return electrode attached to the patient. During arcing, the power source 24 provides an open circuit current of less than thirty milliamps RMS (30 mA_{RMS}) between a distal end 26 of the electrode 22 and the patient's skin. These features allow our device 20 to operate with minimal or no pain or damage to normal healthy skin. The voltage and power are much lower than in the electrical devices used for electrosurgery for cutting and cauterizing, which can be both messy and painful. The lower power, however, also means that our device probably is not suitable for the cutting and cauterizing operations traditionally associated with electrosurgical procedures.

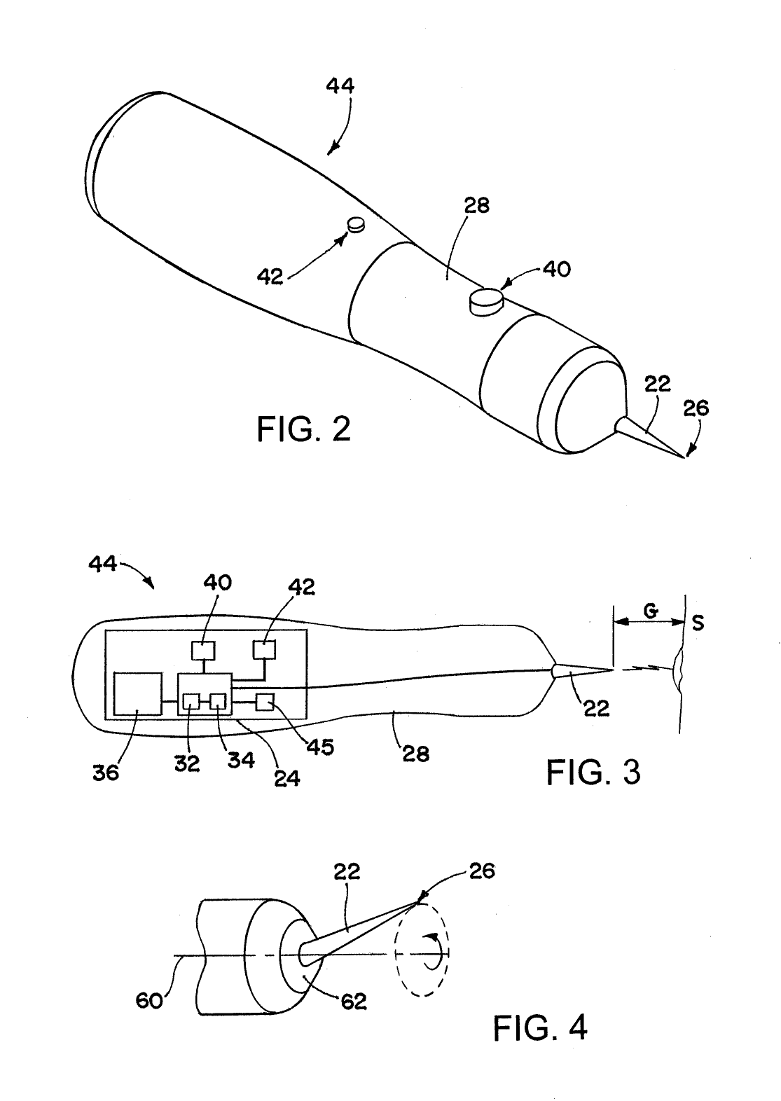

These basic components can be used to build a simple electrical device, an example of which is shown in FIGS. 2 and 3. This electrical device 44 includes an electrode 22 extending from a housing 28, and a power source 24 coupled to the electrode 22 for generating an arc over a gap G when the distal end 26 of the electrode 22 is placed in spaced proximity to a patient's skin S. The power source 24 includes a controller 30, a supply of electricity 36, an on/off switch input device 40 and a status-indicator light output device 42. When an arc is generated, it tends to prefer areas of increased resistance, such as thicker, drier skin, including a callous or a wart, for example, rather than normal skin tissue. The doctor or other operator can move the distal end of the electrode over the problem skin area to be treated. The distal end 26 or tip of the electrode 22 can be brought closer to or into contact with the skin to initiate an arc and moved further away from the skin during normal operation to provide a larger gap for treatment with the arcing electrical spark.

Other features that can be provided in the device 20 are shown in FIG. 1 and include one or more of a motive device 50 or motive means coupled to the electrode 22, a fluid source 52, and a vacuum generator 54. The motive device 50 enables movement of the distal end 26 of the electrode 22 relative to a central longitudinal axis. To provide this movement, the motive device 50 includes a motor 56 controlled by the controller 30 and an associated linkage and/or gearing 58 coupling the motor 56 to the electrode 22. The motive device 50 can move the electrode 22 in any direction, along its axis or another axis, transverse its axis, rotated about an axis, or a combination thereof.

One reason for moving the electrode 22 is to help to initiate and/or maintain the arc. For example, the electrode 22 can be controllably oscillated along its length. To that end the motive device 50 includes oscillating means coupled to the electrode 22 to oscillate the distal end 26 of the electrode within a controlled distance along an axis between an extended position and a retracted position removed from the extended position to facilitate maintaining or reestablishing an arc between the surface of the skin and the distal end 26 of the electrode. The oscillating means includes the controller 30 or a separate control mechanism to control the motive device 50 to control movement of the electrode 22 to the extended position for a first time to make it easier for a spark to jump the gap and initiate an arc and to the retracted position for a second time that is longer than the first time to apply the arc for treatment of a problem skin area. The oscillating means also can include means for monitoring the arc voltage to control the position of the distal end 26 of the electrode. This monitoring function can be incorporated into the device controller 30. By monitoring the voltage, the electrode 22 can be moved automatically to the extended position to re-strike the arc when a voltage drop indicates that the arc has been extinguished.

To apply the electric arc treatment provided by the device to a larger area without the doctor or other operator moving the device 20, the motive device 50 also can move the electrode 22 so that the distal end 26 of the electrode automatically moves through a path or pattern that covers a desired area. The pattern can be circular, linear, zig-zag, or random, for example.

One way to move the electrode 22 in a straight line is to provide a linear slide to which the electrode is affixed for movement in one or more directions transverse the longitudinal axis. When coupled with a device to rotate the electrode 22, a large variety of patterns can be created by moving the electrode linearly and rotatably relative to the axis of rotation.

As shown in FIG. 4, rotating the distal end 26 of the electrode 22 about a longitudinal axis of rotation 60 from which at least a portion of the distal end of the electrode is displaced or offset, without imparting linear motion, can create a circular pattern. The diameter of the circle through which the distal end of the electrode moves can be varied in several ways.

One way to change the diameter is to provide a biasing member 62 coupled to the electrode 22 to bias the electrode toward a central position. This arrangement allows the electrode 22 to move radially outward against the bias force of the biasing member 62 as the electrode rotates. This type of electrode 22 can increase the diameter of the circular pattern through which the distal end 26 moves by increasing the rotational speed. Centrifugal force counters the biasing force to move the distal end 26 outward as a function of the speed. As the rotational speed decreases, the biasing member 62 will urge the electrode 22 back toward a central position and decrease the diameter of the circular path traveled by the distal end 26 of the electrode.

Another way to achieve a similar result is to use an electrode 22 that is flexible transverse its length dimension so that the distal end 26 of the electrode moves radially outward as it rotates, as shown in FIG. 6.

Another way to change the diameter is by offsetting a portion 64 of the electrode 22 toward a distal end 26 relative to another portion 66 of the electrode, and relative to the axis 60 about which the electrode 22 rotates, as shown in FIG. 5. Alternatively, the electrode can be curved, so that the distal end of the electrode is offset from an axis of a proximal portion of the electrode.

To maintain a constant distance, or spark gap, between the distal end 26 of the electrode 22 and the problem area of a patient's skin, the electrode 22 can have a variable length dimension which is increased with increasing rotational speed. The electrode can have telescopic sections or can telescope relative to the housing 28 of the device 20 to extend more or less distance from the housing.

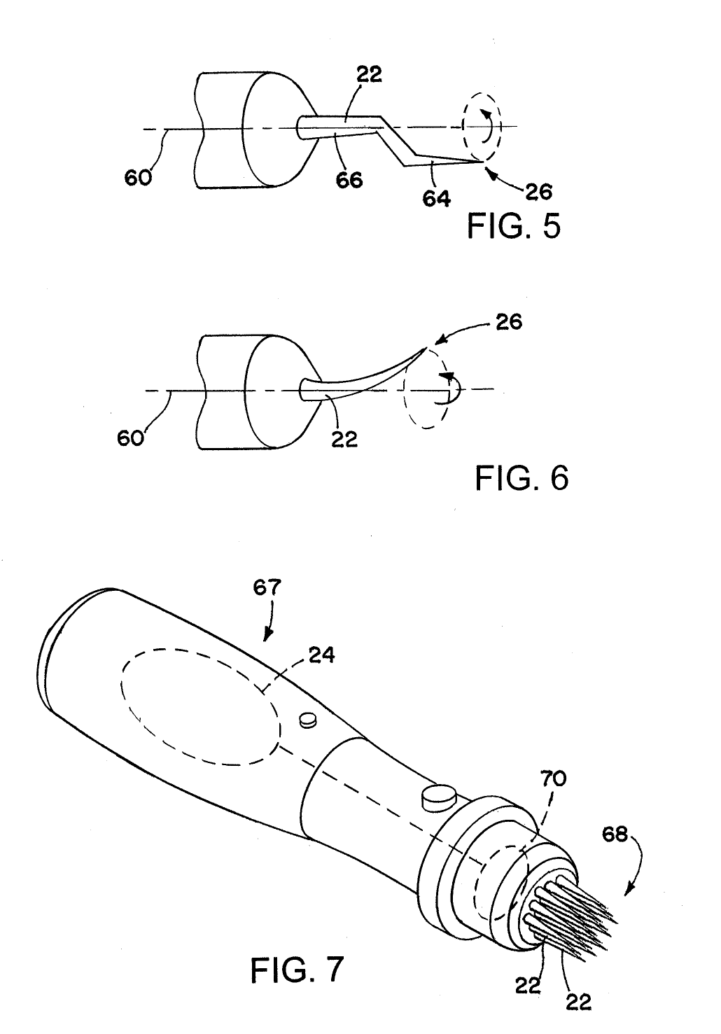

Another way to treat a larger area is to use multiple electrodes, as shown in FIGS. 7-9. In this device 67, the electrode includes an array 68 of electrodes 22 connected to the voltage generator or power source 24, and a voltage distributor 70 for applying a voltage to more than one electrode in the array. The voltage distributor 70 is capable of distributing voltage to multiple electrodes simultaneously or sequentially. The voltage distributor 70 also is capable of distributing voltage to fewer than all of the electrodes.

Returning to FIG. 1, in addition to or in place of the motive device 50, the device 20 can include the fluid source 52 mentioned above. The fluid source 52 includes supply of fluid 76, a fluid pump 78, and an outlet port 80 to direct the fluid toward the distal end 26 of the electrode 22. The supply of fluid 76 includes a reservoir of fluid, such as a liquid and/or gas. The outlet port 80 can be provided by an outlet nozzle extending from the housing 28 to direct fluid toward the distal end 26 of the electrode 22 to facilitate the formation or maintenance of the arc, or provide an additional treatment for the problem skin area.

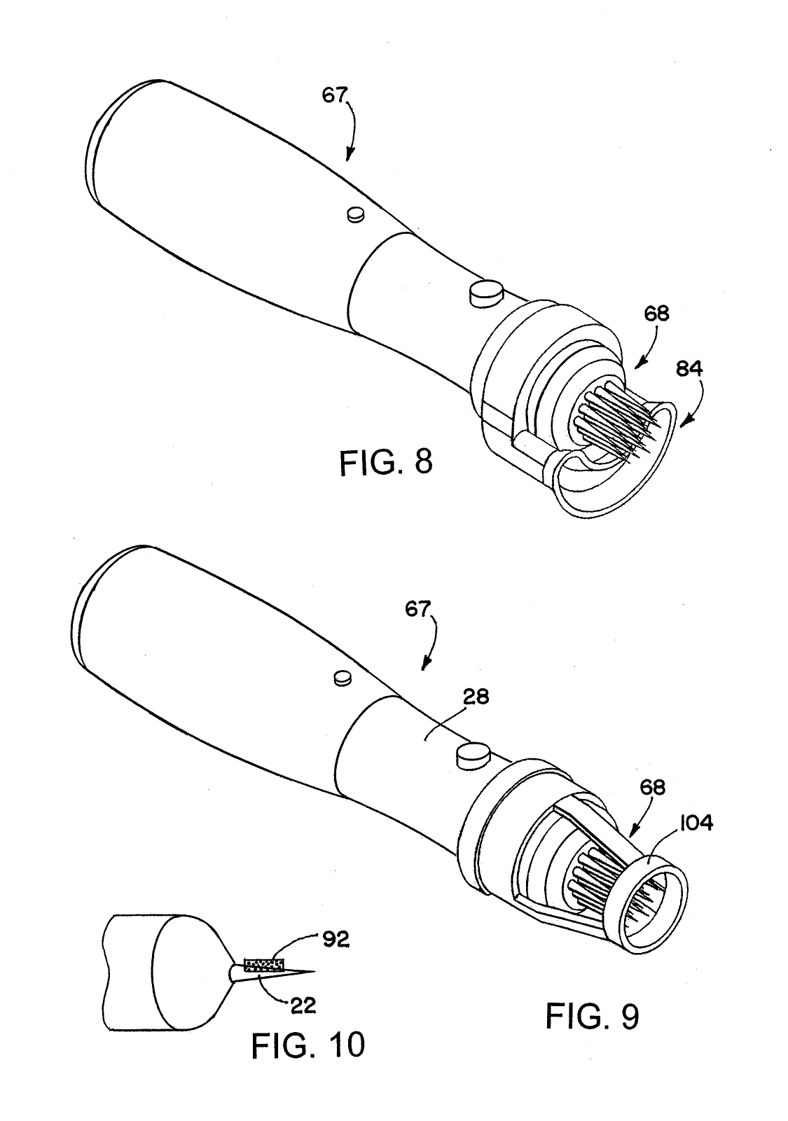

The other added feature in FIG. 1 is the vacuum generator 54 mentioned above. The vacuum generator 54 includes an air pump 82, fan, or other device to create a negative pressure to evacuate air and draw fumes away from the distal end 26 of the electrode 22 and the patient's skin. The illustrated vacuum generator 54 also includes an inlet port 84, an example of which is shown in FIG. 8, and a filter 86, such as charcoal filter media, or another filter media, to filter the fumes in the evacuated air drawn away from the distal end 26 of the electrode. The filter 86 can be replaceable, and can capture particulates, liquids, and/or gases in the evacuated air. The filter thus can absorb odors. The vacuum generator 54 also can be used to recover the fluid from the fluid source 52. The filter can be combined with an electrode so that both the filter and the electrode can be replaced as a unit. One way that this can be accomplished is by incorporating a distal portion of the housing 28 that facilitates interfacing with a receptacle to couple the unit to other components of the device. The device 20 also can include means for introducing a scent into the exhaust air from the air pump 82. For example, the evacuated filtered air can be passed through or past a scented pad 88 or a gel, for example, as it is exhausted from the housing 28 via an exhaust port 90.

In addition to or as an alternative to these features, we also contemplate that the electrode 22 can be removable, such as for cleaning, sterilizing, or replacement. Or as shown in FIG. 10, the electrode 22 can have an abrasive, filed, or otherwise roughened surface 92 mounted to a side of the electrode or integral with the electrode to aid in removal of treated skin where desired.

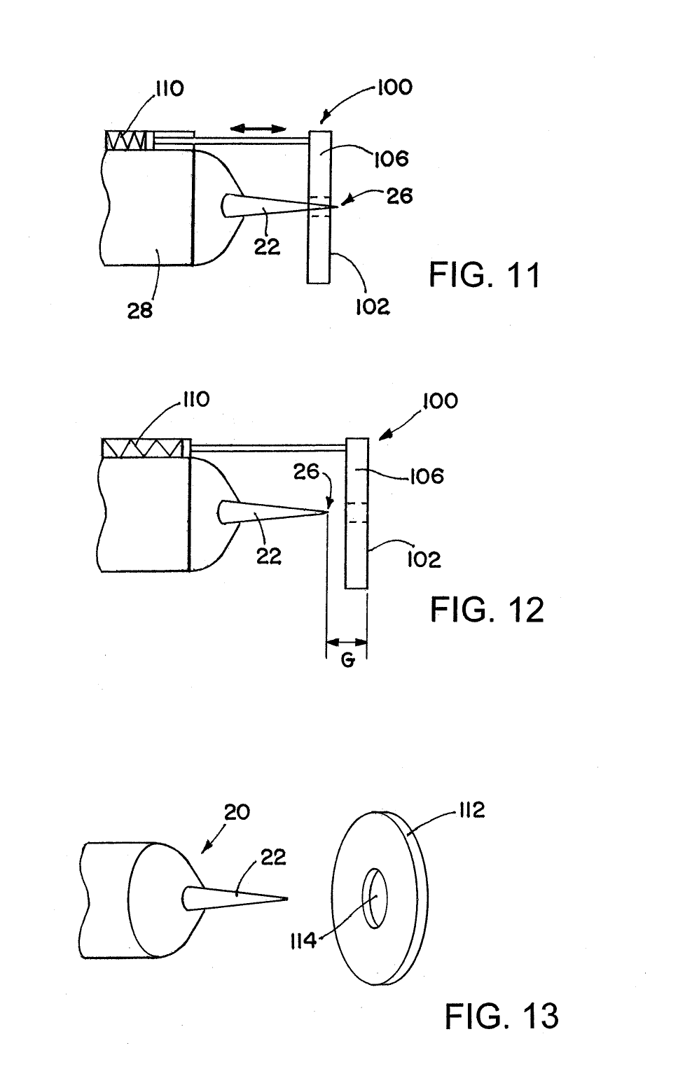

Another feature that can be provided with this device is a non-electrically-conductive spacer 100, shown in FIGS. 11 and 12, for example, that extends beyond the distal end 26 of the electrode 22 for contact with the surface of a patient's skin to define a predetermined gap G between a contact surface 102 of the spacer 100 and the distal end 26 of the electrode. Another exemplary spacer 104 is shown in FIG. 9.

The spacer can be removable and disposable, to ensure sterility between patients, using screws or a snap-fit to hold the spacer on the housing 28, or it can be permanently mounted but movable, for example by being pivotable away from the electrode 22. Being able to move the spacer is advantageous in that moving the spacer out of the way allows the operator to move the distal end 26 of the electrode to contact the skin and initiate an arc, and then use the spacer to define a predetermined gap over which the arc can travel. This predetermined gap can help the operator maintain a consistent arc.

The illustrated spacer 100 includes a movable element 106 that is movable between an extended position to allow the electrode 22 to extend to the contact surface 102 to initiate the electric spark, as shown in FIG. 11, and a retracted position removed from the extended position to space the electrode 22 from the contact surface 102 a distance that provides electrical arcing, as shown in FIG. 12. The illustrated spacer 100 also includes a biasing element 110, such as a spring, that biases the movable element 106 in a distal direction to the extended position.

The spacer is relatively open, or alternatively clear, to maintain visual contact with the distal end 26 of the electrode 22 and to provide visual confirmation of the existence of an electric arc. The transparency required is only such that the doctor or other operator can determine whether the arcing spark is present or has been extinguished. Accordingly, a transparent spacer is not always necessary and a translucent spacer may be suitable for some applications.

We also contemplate using the device we have described in combination with a protective non-electrically-conductive material, in this case a sheet material 112 for placement adjacent a treatment area to protect healthy skin from the electrical arc. The non-electrically-conductive sheet 112 shown in FIG. 13 has an opening 114 for access to the treatment area. The sheet material 112 preferably at least partially surrounds the treatment area. The non-electrically-conductive sheet material 112 may also have a thickness sufficient to space the electrode 22 a distance to provide an optimal electrical arc. The electrical resistance of this material 112 also can help to focus the electrical discharge on the treatment area. The material 112 might have a higher resistance than the treatment area, for example. The sheet material 112 also can protect the treated area after treatment, when the treated area might be more sensitive. This combination can be considered a kit, including both the electrical device 20 and the non-electrically-conductive material, which also can function as a bandage.

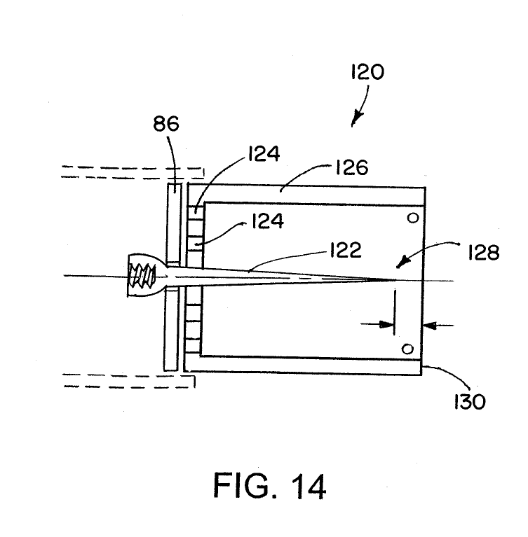

FIG. 14 shows an exemplary removable electrode assembly unit 120 that includes an electrode 122 having a threaded base, a filter 86 and a housing or shroud 126 that includes one or more passages 124 that define an inlet port 84 for drawing air from around the electrode through the filter 86. The shroud 126 also serves as a spacer to space a distal tip 128 of the electrode 122 relative to a contact surface 130 at a distal end of the shroud 126. Instead of or in addition to the threaded connection shown, the electrode 122 and/or the shroud 126 can be secured to a distal end of the housing 28 (FIG. 1), and in fact the shroud 126 can define a distal portion of the housing 28. The shroud 126 and/or the electrode 122 can be secured in place for use by a threaded, snap, or press-fit connection that allows for the removal and replacement of the electrode assembly 120 as a unit.

Our invention also provides a method for treating problem skin areas that includes the following steps: (a) generating a voltage and providing that voltage to a distal end of an electrode; and (b) positioning the electrode in proximity to a patient's skin to form an arcable gap between the electrode and the skin to produce an electric spark that arcs across the gap with sufficient intensity to treat the problem skin area but insufficient to cause significant damage to normal surrounding tissue. The generating step includes generating a high frequency (at least 100 kHz) voltage (less than 2 kV_{RMS} open circuit) with less than 2 W of power to a monopolar electrode. The generating step also can include providing a voltage such that in the moving step the electric spark arcs across the gap with a substantially constant open circuit current of less than thirty milliamps RMS (30 mA_{RMS}) between the electrode and the skin.

When employing a spacer, the positioning step includes contacting the surface of a patient's skin near a problem skin area with a non-electrically-conductive element that spaces the patient's skin from a distal end of an electrode to form a gap between a contact surface of the non-electrically conductive element and the distal end of the electrode.

When the device includes a vacuum source, the method can include the step of generating a vacuum near the patient's skin to evacuate air and draw fumes away from the patient's skin, and/or filtering fumes from the evacuated air drawn from the patient's skin.

As mentioned above, in the multiple-electrode embodiment of the device, fewer than all of the electrodes can be energized. Accordingly, the method can include the steps of: (a) measuring the size of a treatment area of a patient's skin; and (b) selecting a plurality of electrodes to distribute voltage to based on the measured size of the treatment area. In this way, the problem skin area can be treated all at once or in less time than if a single electrode had to be moved over the same area.

Although the invention has been shown and described with respect to certain embodiments, equivalent alterations and modifications will occur to others skilled in the art upon reading and understanding this specification and the annexed drawings. In particular regard to the various functions performed by the above described integers (components, assemblies, devices, compositions, etc.), the terms (including a reference to a “means”) used to describe such integers are intended to correspond, unless otherwise indicated, to any integer which performs the specified function of the described integer (i.e., that is functionally equivalent), even though not structurally equivalent to the disclosed structure which performs the function in the herein illustrated exemplary embodiment of the invention.

1. An electrical device that is used to treat problem skin areas, including warts, comprising:
(a) an electrode; and
(b) a power source coupled to the electrode for providing an arc over a gap between a distal end of the electrode and a patient's skin when the electrode is placed in spaced proximity to a patient's skin.

2. The device of claim 1, where the power source provides electricity to the electrode with a frequency of at least 100 kHz, an open-circuit voltage of less than 2 kVRMS, and a total power of less than 2 W.

3. The device of claim 1, without a return electrode.

4. The device of claim 1, where the electrode is removable.

5. The device of claim 1, comprising a non-electrically-conductive spacer extending beyond the distal end of the electrode for contact with the surface of a patient's skin to define a predetermined gap between a contact surface of the spacer and the distal end of the electrode.

6. The device of claim 5, where the spacer is removable.

7. The device of claim 5, where the spacer includes a movable element that is movable between an extended position to allow the electrode to extend to the contact surface to initiate the electric spark and a retracted position removed from the extended position to space the electrode from the contact surface a distance that provides electrical arcing.

8. The device of claim 7, where the movable element is biased in a distal direction.

9. The device of claim 7, where the spacer is relatively clear to maintain visual contact with the distal end of the electrode and to provide visual confirmation of the existence of an electric arc.

10. The device of claim 1, where the electrode has an abrasive surface to aid in removal of treated skin.

11. The device of claim 1, comprising oscillating means coupled to the electrode to oscillate the distal end of the electrode within a controlled distance along an axis between an extended position and a retracted position removed from the extended position to facilitate maintaining or re-establishing an arc between the surface of the skin and the distal end of the electrode.

12. The device of claim 11, where the oscillating means controls the electrode to move to the extended position for a first period of time and to the retracted position for a second period of time that is longer than the first period of time.

13. The device of claim 11, where the oscillating means monitors the arc voltage to control the position of the distal end of the electrode, and moves the electrode to the extended position to re-strike the arc when a voltage drop indicates that the arc has been extinguished.

14. The device of claim 1, comprising a vacuum generator to evacuate air and draw fumes away from the distal end of the electrode and the patient's skin.

15. The device of claim 14, where the vacuum generator includes a filter to filter the fumes in the evacuated air drawn away from the distal end of the electrode.

16. The device of claim 14, where the filter is replaceable.

17. The device of claim 16, where the replaceable filter is coupled with a replaceable electrode for replacement as a unit.

18. The device of claim 14, where the evacuated air is passed through a filter media to eliminate odors.

19. The device of claim 14, where the evacuated filtered air is passed through a scented medium before being exhausted.

20. The device of claim 1, where the electrode has a length dimension, and the distal end of the electrode is movable relative to a central longitudinal axis.

21. The device of claim 20, where the electrode is rotatable about the longitudinal axis to allow the distal end of the electrode to sweep in a circular pattern.

22. The device of claim 20, where the rotational speed determines the diameter of the circular pattern, generating an increasing diameter with increasing speed.

23. The device of claim 22, where the electrode has a variable length dimension which is increased with increasing rotational speed to maintain a constant distance between the electrode and a problem area of a patient's skin.

24. The device of claim 20, where the device includes one or more of:
(a) an electrode that is affixed to a linear slide for movement transverse the longitudinal axis;
(b) an electrode that is flexible transverse its length dimension and is rotatable about its length so that the distal end of the electrode moves radially outward as it rotates; and
(c) a biasing member coupled to the electrode to bias the electrode toward a central position and allows the electrode to move radially outward against the biasing member as the electrode rotates.

25. The device of claim 1, where the electrode has a length dimension, and a portion at a distal end of the electrode is offset from a longitudinal axis of another portion of the electrode.

26. The device of claim 25, including motive means coupled to the electrode for rotating the electrode about the longitudinal axis, causing the distal end of the electrode to travel in a circular pattern.

27. The device of claim 1, comprising a supply of gas or liquid and an outlet port to direct the fluid toward a distal end of the electrode.

28. The device of claim 1, in combination with a protective non-electrically-conductive material for placement adjacent a treatment area to protect healthy skin from the electrical arc.

29. The combination of claim 28, where the non-electrically-conductive material at least partially surrounds the treatment area.

30. The combination of claim 28, where the distal end of the device interacts with the non-electrically-conductive material to space the electrode a predetermined distance to provide an optimal electrical arc.

31. The device of claim 1, where the electrode includes an array of electrodes connected to the voltage generator; and a voltage distributor for applying a voltage to more than one electrode in the array.

32. The device of claim 31, where the voltage distributor is capable of distributing voltage to multiple electrodes simultaneously or sequentially.

33. The device of claim 31, where the voltage distributor is capable of distributing voltage to fewer than all of the electrodes.

34. A method for treating problem skin areas, including warts, comprising the following steps:
(a) generating a voltage and providing that voltage to a distal end of an electrode; and
(b) positioning the electrode in proximity to a patient's skin to form an arcable gap between the electrode and the skin to produce an electric spark that arcs across the gap with sufficient intensity to treat the problem skin area but insufficient to cause significant damage to normal surrounding tissue.

35. The method of claim 34, where the generating step includes generating a high frequency (at least 100 kHz) voltage (less than 2 kVRMS open circuit) with less than 2 W of power to a monopolar electrode.

36. The method of claim 34, where the generating step includes providing a voltage such that in the moving step the electric spark arcs across the gap with a substantially constant current of less than 30 mARMS between the electrode and the skin.

37. The method of claim 34, where the positioning step includes contacting the surface of a patient's skin near a problem skin area with a non-electrically-conductive element that spaces the patient's skin from a distal end of an electrode to form a gap between a contact surface of the non-electrically conductive element and the distal end of the electrode.

38. The method of claim 34, comprising the step of generating a vacuum near the patient's skin to evacuate air and draw fumes away from the patient's skin.

39. The method of claim 38, comprising the step of filtering fumes from the evacuated air drawn from the patient's skin.

40. The method of claim 34, comprising the steps of:
(a) measuring the size of a treatment area of a patient's skin; and
(b) selecting a plurality of electrodes to distribute voltage to based on the measured size of the treatment area.
